(19) United States
(12) Patent Application Publication
(10) Pub. No.: US 2008/0214950 A1
(43) Pub. Date: Sep. 4, 2008

(54) PROSTATE CANCER DETECTION APPARATUS

(76) Inventors: Jens Fehre, Hausen (DE); Ralf Nanke, Neunkirchen am Brand (DE)

Correspondence Address: SCHIFF HARDIN, LLP;PATENT DEPARTMENT, 6600 SEARS TOWER, CHICAGO, IL, 60606-6473, US

(21) Appl. No.: 12/038,933

(22) Filed: Feb. 28, 2008

(30) Foreign Application Priority Data
Mar. 1, 2007 (DE) 10 2007 010 046.0

(51) Int. Cl. A61B 1/307; A61B 5/01; A61B 7/00

(52) U.S. Cl. 600/549; 600/135; 600/586

(57) ABSTRACT

A prostrate cancer detection apparatus has a measurement sensor for detection of at least one prostate cancer-specific biomarker, an evaluation unit for determination of at least one risk value from at least one prostate cancer-specific biomarker detected by the measurement sensor, and a display unit to display the at least one determined risk value.

### BACKGROUND OF THE INVENTION

1. Field of the Invention

The present invention concerns a prostate cancer detection apparatus.

2. Description of the Prior Art

Prostate cancer (Prostate cancer) exhibits a rising incidence and is the most common cancer in men over 50 years of age in the industrial nations. However, very good recovery chances exist given early detection.

Presently DRU (digital rectal examination, palpation) and PSA tests (prostate-specific antigen, blood test) as well as TRUS (trans-rectal ultrasound) are used for the early detection of prostate cancers.

These methods individually and in combination exhibit only an insufficient sensitivity and specificity for prostate cancers. With regard to prostate cancer preventative examinations there is presently no preventative examination that is covered by health insurance (as with mammography examinations for cancer prevention in women, for example). The costs are only borne by the health insurance given a basis for suspicion of prostate cancer; otherwise only the possibility of self-payment by the patient exists, as is already the case in the majority of PSA tests.

An examination for the presence of prostate cancer by means of magnetic resonance imaging is described in US 2004/0254445 A1. In this examination a contrast agent can also be used that accumulates in the prostate. Due to the high acquisition and operating costs of a magnetic resonance apparatus, this examination is not suitable for a commonplace prostate cancer examination.

A portable prostate cancer detection apparatus that has a reaction cell in which a tissue sample is subjected to an electrochemical analysis (in vitro diagnosis) is known from US 2004/0053425 A1 and US 2004/0072263 A1. However, this tissue sample must first be invasively extracted, for example by a biopsy.

### SUMMARY OF THE INVENTION

An object of the present invention is to provide a simple prostate cancel detection apparatus for a certain detection of prostate cancer.

The above object is inventively achieved by a prostate cancer detection apparatus having a measurement sensor for detection of at least one prostate cancer-specific biomarker, an evaluation unit; for determination of at least one risk value from at least one prostate cancer-specific biomarker detected by the measurement sensor, and a display unit that displays the at least one determined risk value.

A regular prostate cancer preventative examination in men can be conducted by the inventive prostate cancer detection apparatus. At least one prostate cancer-specific biomarker that accumulates in the appertaining areas of the prostate must merely be administered to the patient for this.

The administration of a prostate cancer-specific biomarker can ensue via a direct injection into the prostate. A systemic (intravenous) or oral administration is likewise possible.

The biomarker (combination of a molecular marker such as, for example, a specific protein or antibody and a “detection component” such as, for example, a bubble or a magnetic particle) detectable by the prostate cancer detection apparatus according to the invention bonds exclusively with specific molecules which are specific to prostate cancer or, respectively, the association angiogenesis (formation of new blood vessels from already existing blood vessels) (“molecular tracking”).

The detection of at least one prostate cancer-specific biomarker by means of the inventive prostate cancer detection apparatus exhibits a high sensitivity and specificity. Due to the spatial proximity to the prostate during the examination, both the intracorporeal measurement sensor and the extracorporeal measurement sensor ensure a reliable measurement of the concentration of existing prostate cancer-specific biomarkers and (resulting from this) a reliable determination of a prostate cancer risk.

The inventive prostate cancer detection apparatus is simple to use and can be used ubiquitously by established physicians [physicians in private practice] due to the low costs.

Moreover, no radiation exposures (as with x-ray examinations, for example) occur in examinations that are executed with the inventive prostate cancer detection apparatus.

The examination method that can be implemented with the prostate cancer detection apparatus according to claim 1 is thus suitable for a systematic early detection of the prostate cancer with which a reliable and cost-effective prostate cancer screening is possible in a large population.

If at least one of the risk values determined by the evaluation unit exceeds a predetermined limit value, then a positive finding exists and the patient is transferred to a clinic for urological examination. Complicated examinations can then be implemented in the clinic, for example a prostate biopsy or the magnetic resonance examination described in US 2004/0254445 A1.

A biomarker that is detectable by the prostate cancer detection apparatus described in claim 1 exhibits specific physical and/or chemical properties that enable its detection in the prostate. For example, a biomarker suitable for the inventive prostate cancer detection apparatus can comprise magnetic particles or generate heat via chemical reactions. In the framework of advantageous embodiments, the measurement sensor of the inventive prostate cancer detection apparatus comprises for this a magnet sensor (detection of magnetic signals) and/or a temperature sensor (detection of thermal signals) and/or an optical sensor (detection of luminescent biomarkers) and/or an acoustic sensor (detection of acoustic signals emitted by a biomarker excited via ultrasound, “targeted bubbles”), for example.

In accordance with the invention it is possible to execute the measurement sensor as an intracorporeal measurement sensor or—given sufficient strength of the signals of the prostate cancer-specific biomarker—as an extracorporeal measurement sensor. An extracorporeal measurement sensor is hereby to be placed on the body surface and optimally near the prostate.

Since the accumulation in the prostate of at least one prostate cancer biomarker is detected by the inventive prostate cancer biomarker, it is particularly advantageous when the measurement sensor is fashioned as an intracorporeal measurement sensor. In this case the detection of a slight accumulation of a prostate cancer-specific biomarker in the prostate is also reliably possible.

According to a preferred embodiment of the prostate cancer detection apparatus, the intracorporeal measurement sensor is fashioned as a single-use article. This represents an effective prevention against risk of infection. Moreover, the sterilization effort is saved, whereby the patient throughput given serial examinations can be distinctly increased.

### BRIEF DESCRIPTION OF THE DRAWINGS

FIG. 1 shows a first embodiment of the inventive prostate cancer detection apparatus in a schematic section view.

FIG. 2 shows a second embodiment of the inventive prostate cancer detection apparatus in a schematic section view.

### DESCRIPTION OF THE PREFERRED EMBODIMENTS

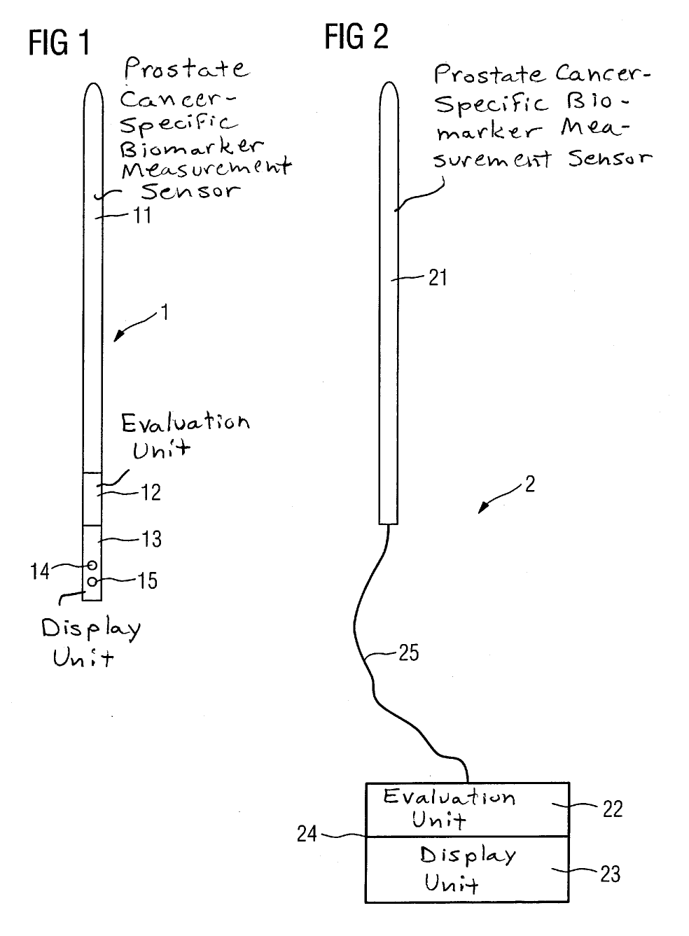

A prostate cancer detection apparatus 1 with a measurement sensor 11 for detection of at least one prostate cancer-specific biomarker is shown in FIG. 1. The measurement sensor 11 is executed as an intracorporeal measurement sensor in the exemplary embodiment.

The prostate cancer detection apparatus 1 furthermore comprises an evaluation unit 12 for determination of at least one risk value from at least one prostate cancer-specific marker detected by the intracorporeal measurement sensor 11 as well as a display unit 13 for display of at least one determined risk value.

In the exemplary embodiment shown in FIG. 1 the evaluation unit 12 and the display unit 13 are integrated in the intracorporeal measurement sensor 11.

Due to the resulting small structural size for the evaluation unit 12 and the display unit 13, the display unit 13 exhibits only a small display. Light-emitting diodes are particularly suitable for this purpose. For example, a green LED 14 illuminates given a negative prostate cancer finding (thus positive for the patient) and a red LED 15 illuminates given suspicion of prostate cancer, i.e. given a possible positive finding.

The intracorporeal measurement sensor 11 can be executed as a single-use article. It is then advantageous to execute the display unit 13 or the evaluation unit 12 and the display unit 13 such that it can be removed or detached and therewith reused again.

In accordance with the invention it is also possible for the evaluation unit 12 to functionally interact with an external display unit. The evaluation unit 12 is then to be coupled with a display unit (not shown in FIG. 1) after the examination. In a further embodiment, the evaluation unit 12 can be executed only as a memory unit (not shown in FIG. 1) in which the determined values of a biomarker concentration are merely stored. The stored values of the biomarker concentration can then be read out in a readout unit and fed to the evaluation unit after the end of the examination.

A further prostate cancer detection apparatus that is designated with 2 is shown in FIG. 2. The prostate cancer detection apparatus 2 likewise comprises a measurement sensor 21 for detection of at least one prostate cancer-specific biomarker. In this exemplary embodiment the measurement sensor 21 is executed as an intracorporeal measurement sensor.

The prostate cancer detection apparatus 2 furthermore comprises an evaluation unit 22 for determination of at least risk value from at least one prostate cancer-specific biomarker detected by the intracorporeal measurement sensor 21 as well as a display unit 23 for display of at least one determined risk value.

In contrast to the integrated design according to FIG. 1, in the prostate cancer detection apparatus according to FIG. 2 the evaluation unit 22 and the display unit 23 are executed as separate components that are advantageously arranged in a common housing. Due to a larger structural volume relative to an embodiment according to FIG. 1, the display does not necessarily have to be executed as an LED display. Rather, the display can also be fashioned as an analog or digital display (LCD). A monitor is also possible as a display in the framework of the invention.

In the variant shown in FIG. 2 the intracorporeal measurement sensor 21 is connected with the evaluation unit 22 via a data and power supply cable 25.

In the prostate cancer detection apparatus 2 the intracorporeal measurement sensor 21 can also be executed as a single-use article. However, in accordance with the invention the intracorporeal measurement sensor 21 can also be designed as a reusable measurement sensor. For this the measurement sensor 21 can be detached from the data and supply cable 25 for disposal or, respectively, for sterilization.

An acoustic signal can be provided as an alternative to an optical display (LEDs 14 and 15, analog or digital display) or to support an optical display.

As is apparent from the specification of the exemplary embodiments, the inventive prostate cancer detection apparatus is simple to use and can be used extensively by physicians in private practice due to the low costs.

The examination method that is implementable with the prostate cancer detection apparatus according to the invention is thus suitable for a systematic early detection of prostate cancer with which a reliable and cost-effective prostate cancer screening is possible in a large population.

Although modifications and changes may be suggested by those skilled in the art, it is the intention of the inventors to embody within the patent warranted hereon all changes and modifications as reasonably and properly come within the scope of their contribution to the art.

We claim as our invention:

1. A prostrate cancer detection apparatus comprising:
a measurement sensor configured to interact with an examination subject to detect at least one prostrate cancer-specific biomarker;
an evaluation unit supplied with said at least one prostrate cancer-specific biomarker detected by said measurement sensor that determines at least one risk value from said at least one prostrate cancer-specific biomarker; and
a display unit in communication with said evaluation unit that presents a visual display of said at least one predetermined risk value.

2. A prostrate cancer detection apparatus as claimed in claim 1 wherein said measurement sensor is an intracorporeal measurement sensor.

3. A prostrate cancer detection apparatus as claimed in claim 2 wherein said extracorporeal measurement sensor is an endoscope.

4. A prostrate cancer detection apparatus as claimed in claim 2 wherein said intracorporeal measurement sensor has a cylindrical shape.

5. A prostrate cancer detection apparatus as claimed in claim 2 wherein said intracorporeal measurement sensor has a tubular shape.

6. A prostrate cancer detection apparatus as claimed in claim 1 wherein said measurement sensor is an extracorporeal measurement sensor.

7. A prostrate cancer detection apparatus as claimed in claim 1 wherein said measurement sensor comprises a magnetic sensor.

8. A prostrate cancer detection apparatus as claimed in claim 1 wherein said measurement sensor comprises a temperature sensor.

9. A prostrate cancer detection apparatus as claimed in claim 1 wherein said measurement sensor comprises an optical sensor.

10. A prostrate cancer detection apparatus as claimed in claim 1 wherein said measurement sensor comprises an acoustic sensor.

11. A prostrate cancer detection apparatus as claimed in claim 1 wherein said measurement sensor is a single-use article.

12. A prostrate cancer detection apparatus as claimed in claim 1 wherein said display unit displays said risk value in an analog presentation.

13. A prostrate cancer detection apparatus as claimed in claim 1 wherein said display unit displays said risk value in a digital presentation.

14. A prostrate cancer detection apparatus as claimed in claim 1 wherein said display unit displays said risk value in a limit value display format.

15. A prostrate cancer detection apparatus as claimed in claim 1 wherein said evaluation unit and said display unit are separate units from each other.

16. A prostrate cancer detection apparatus as claimed in claim 1 wherein said evaluation unit and said display unit are integrated together as a common unit.

17. A prostrate cancer detection apparatus as claimed in claim 1 wherein said measurement sensor, said evaluation unit and said display unit are integrated in a common, single, unitary component.

18. A prostrate cancer detection apparatus as claimed in claim 1 wherein said measurement sensor is separate from said evaluation unit, and comprising a communication cable connecting said measurement sensor to said evaluation unit to supply a signal representing said at least one prostrate cancer-specific biomarker from said measurement sensor to said evaluation unit.

19. A prostrate cancer detection apparatus as claimed in claim 18 wherein said measurement sensor is detachable from said communication cable.
